(19) United States
(12) Patent Application Publication
(10) Pub. No.: US 2008/0076978 A1
(43) Pub. Date: Mar. 27, 2008

(54) SYSTEM, APPARATUS, METHOD, AND PROGRAM PRODUCT FOR MEASURING BIOLOGICAL INFORMATION

(75) Inventors: Kazushige Ouchi, Kanagawa (JP); Takuji Suzuki, Kanagawa (JP); Kenichi Kameyama, Kanagawa (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Kabushiki Kaisha Toshiba, Tokyo (JP)

(21) Appl. No.: 11/688,500

(22) Filed: Mar. 20, 2007

(30) Foreign Application Priority Data
Sep. 25, 2006 (JP) 2006-259298

(51) Int. Cl. A61B 5/00

(52) U.S. Cl. 600/301

(57) ABSTRACT

An apparatus for measuring biological information of a user, the apparatus includes a sensor that measures the biological information; a storing unit that stores a measurement result obtained by the sensor; a communication state recognizing unit that recognizes a current communication state; a determining unit that determines a transmission timing capable of transmitting data to an information terminal, based on the communication state recognized by the communication state recognizing unit; and a transmitting unit that transmits the measurement result stored in the storing unit to the information terminal at the transmission timing determined by the determining unit.

### CROSS-REFERENCE TO RELATED APPLICATIONS

This application is based upon and claims the benefit of priority from the prior Japanese Patent Application No. 2006-259298, filed on Sep. 25, 2006; the entire contents of which are incorporated herein by reference.

### BACKGROUND OF THE INVENTION

1. Field of the Invention

The present invention relates to a system, an apparatus, a method, and a program product for measuring biological information that are used for measuring biological information of users.

2. Description of the Related Art

Conventionally, attempts have been made to measure biological information using exclusive-use terminals such as blood pressure monitors for home-use and body fat scales, and made to use the measurement results for maintaining good health. However, most of these conventional devices specialize in only measuring the data and have no functions to collaborate with other devices. Thus, the users are required to manage the measured data by themselves.

Some measuring devices have a function to collaborate with another device like a personal computer (PC) that manages data. However, in these situations, the user needs to connect an exclusive-use terminal to the device and also needs to operate a data management application stored in the PC. Thus, it requires time and effort from the user.

As another example, there is a service that manages users' biological information on the web or the like and provides an appropriate advice and the like. However, in principle, this type of service requires that the users manually input their biological information and puts a burden on the users. In addition, there is a possibility that the users forget to input their biological information. Thus, it may not be possible to manage the data with convenience in some cases.

To cope with these situations, other attempts have been made to have a device used for measuring biological information collaborate with another device used for managing data such as a PC or a portable terminal, so that the measurement results are semi-automatically managed. One example of such attempts is disclosed as a technique to connect a plurality of measuring devices to a PC via a receiver so that the PC collectively manages the measured data, and further, an advice on a health and the like are provided via the Internet as a result of the PC's collaborating with a data server (see, for example, JP-A No. 2004-283570 (KOKAI). Another example is disclosed as a technique to transmit and manage data regardless of the location, as a result of a mobile phone's collaborating with a wearable sensor (see, for example, JP-A No. 2004-147705 (KOKAI).

The technique disclosed in JP-A No. 2004-283570 (KOKAI), however, requires that the user push a transmission button to transmit the data. The technique disclosed in JP-A No. 2004-147705 (KOKAI) requires that the user perform an operation to transmit the data from the sensor to the mobile phone and to upload the data to a health management server.

It is preferable if a service that constantly measures and manages biological information on a daily basis is designed so as to make the users' burden as light as possible. However, a function that automatically uploads the data for the purpose of reducing the users' burden has the disadvantage of uploading data that is not worth transmitting (e.g. data that has not correctly been measured, data that has been measured in too short a period of time, data that has low importance). As a result, such a function results in a waste of communication costs and an increase in the load on the data management side.

Also, in such a system that is constantly used, keeping the sensor and the management terminal in such a state that they are able to wirelessly communicate with each other at all times causes a problem related to electric power consumption, especially on the sensor side. In addition, the sensor and the management terminal need to maintain a positional relationship so that they are able to wirelessly communicate with each other. If the communication between them is disconnected for some reason, it is necessary to address the problem of the data that fails to be transmitted.

### SUMMARY OF THE INVENTION

According to one aspect of the present invention, a biological information measuring system includes a measuring apparatus that measures biological information of a user; and an information terminal that manages the biological information measured by the measuring apparatus, wherein the measuring apparatus includes: a sensor that measures the biological information; a first storing unit that stores a measurement result obtained by the sensor; a recognizing unit that recognizes a current communication state of communication between the measuring apparatus and the information terminal; a determining unit that determines a transmission timing capable of transmitting data to the information terminal, based on the communication state recognized by the communication recognizing unit; and a first transmitting unit that transmits the measurement result stored in the first storing unit to the information terminal at the transmission timing determined by the determining unit, and the information terminal includes: a first receiving unit that receives the measurement result from the measuring apparatus; and a first analyzing unit that analyzes the measurement result received by the first receiving unit.

According to another aspect of the present invention, An apparatus for measuring biological information of a user, the apparatus includes a sensor that measures the biological information; a storing unit that stores a measurement result obtained by the sensor; a communication state recognizing unit that recognizes a current communication state; a determining unit that determines a transmission timing capable of transmitting data to an information terminal, based on the communication state recognized by the communication state recognizing unit; and a transmitting unit that transmits the measurement result stored in the storing unit to the information terminal at the transmission timing determined by the determining unit.

According to still another aspect of the present invention, a measuring method in a biological information measuring system including a measuring apparatus that measures biological information of a user and an information terminal that manages the biological information measured by the measuring apparatus, the measuring method includes measuring the biological information by using a sensor included in the measuring apparatus; recognizing a current communication state by using the measuring apparatus; determining a transmission timing capable of transmitting data to the information terminal by using the measuring apparatus, based on the communication state; transmitting a measurement result stored in a storing unit that stores the measurement result obtained by the sensor by using the measuring apparatus, to the information terminal at the transmission timing; receiving the measurement result from the measuring apparatus by using the information terminal; and analyzing the measurement result by using the information terminal.

A computer program product according to still another aspect of the present invention causes a computer to perform the method according to the present invention.

### BRIEF DESCRIPTION OF THE DRAWINGS

FIG. 1 is a block diagram of an overall biological-information measuring system 1 according to a first embodiment of the present invention;

FIG. 2 is a drawing for explaining a data format of user state data;

FIG. 3 is a drawing of an exterior appearance of a biological-information measuring apparatus 10;

FIG. 4 is a flowchart of a process performed by the biological-information measuring apparatus 10;

FIG. 5 is a flowchart for explaining the details of a transmission timing judging process (step S110);

FIG. 6 is a flowchart of a process performed by an information terminal 20;

FIG. 7 is a diagram of a relevant hardware configuration of the biological-information measuring apparatus 10 according to the first embodiment;

FIG. 8 is a block diagram of an overall biological information measuring system 2 according to a second embodiment of the present invention;

FIG. 9 is a drawing for explaining a data format of biological information;

FIG. 10 is a flowchart of a process performed by a biological-information measuring apparatus 11 according to the second embodiment;

FIG. 11 is a block diagram of an overall biological information measuring system 3 according to a first modification example of the second embodiment;

FIG. 12 is a block diagram of a biological information measuring system 4, in its entirety, according to a second modification example of the second embodiment;

FIG. 13 is a block diagram of an overall biological information measuring system 5 according to a third embodiment of the present invention;

FIG. 14 is a drawing for explaining the data structure of an importance level table stored in an importance-level-table storing unit 220;

FIG. 15 is a block diagram of an overall biological information measuring system 6 according to a fourth embodiment of the present invention;

FIG. 16 is a flowchart of a call receiving process performed by an information terminal 23 according to the fourth embodiment;

FIG. 17 is a flowchart for explaining the details of the transmission timing judging process (step S110) performed by the biological-information measuring apparatus 11 according to the fourth embodiment;

FIG. 18 is a flowchart of a call receiving process performed by the information terminal 23 included in the biological information measuring system 6 according to a first modification example of the fourth embodiment;

FIG. 19 is a block diagram of a biological information measuring system 7, in its entirety, according to a fifth embodiment of the present invention; and

FIG. 20 is a drawing for explaining the data structure of a call-receiving-mode determination table included in a call-receiving-operation controlling unit 240.

### DETAILED DESCRIPTION OF THE INVENTION

Exemplary embodiments of a biological information measuring system, a measuring apparatus, a biological information measuring method, and a biological information measuring program product according to the present invention will be explained in detail, with reference to the accompanying drawings. It should be noted that the present invention is not limited to these exemplary embodiments.

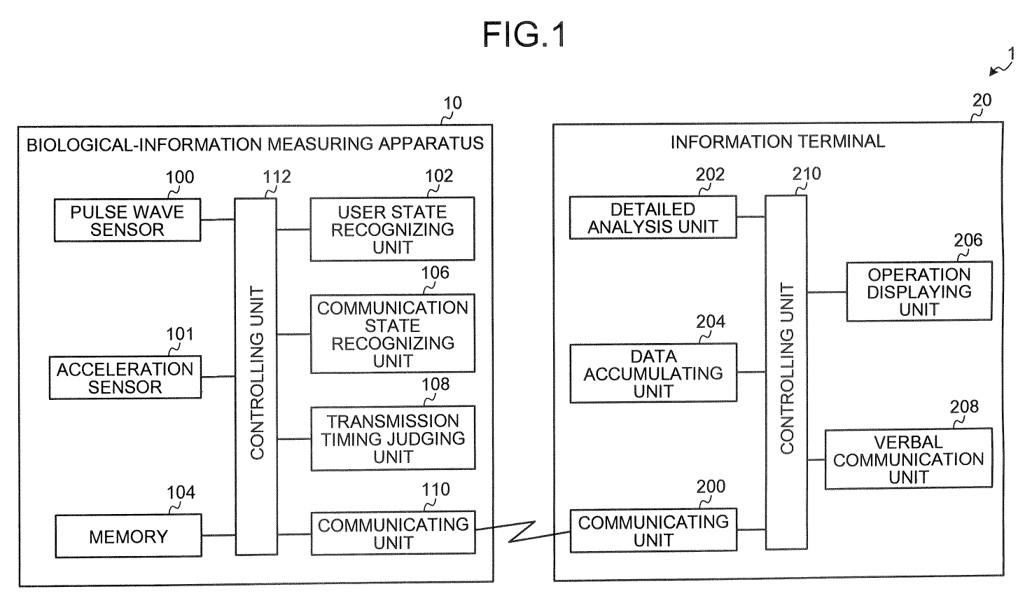

As shown in FIG. 1, a biological information measuring system 1 according to a first embodiment of the present invention includes a biological-information measuring apparatus 10 and an information terminal 20. The biological-information measuring apparatus 10 includes a pulse wave sensor 100, an acceleration sensor 101, a user state recognizing unit 102, a memory 104, a communication state recognizing unit 106, a transmission timing judging unit 108, a communicating unit 110, and a controlling unit 112.

The pulse wave sensor 100 and the acceleration sensor 101 measure biological information of a user. The pulse wave sensor 100 measures pulse waves as the biological information. The acceleration sensor 101 measures, as the biological information, accelerations in the directions of three axes, namely, the x-axis, the y-axis, and the z-axis. In some situations, depending on the purpose for which the biological information is used, only data from one of the pulse wave sensor 100 and the acceleration sensor 101 is needed. In such situations, only the type of data that is needed is measured. For example, when the purpose for which the biological information is used is to judge sleeping conditions, e.g. Rapid Eye Movement (REM) sleep or non-REM sleep, acceleration data is not necessary. Thus, the accelerations do not need to be measured.

The types and the number of the sensors to be used are not limited to the example described in the exemplary embodiments. Any type of sensors and any number of sensors may be used as long as they detect information obtained from a living body of a user.

The user state recognizing unit 102 recognizes a user state, based on the biological information measured by the pulse wave sensor 100 and the acceleration sensor 101. According to the first embodiments pulse wave interval data and movement amount data are obtained as the user state data. The pulse wave interval data includes values that serve as an indicator for the state of autonomic nerves of a user, such as sympathetic nerves and parasympathetic nerves. It is possible to determine the level of relaxation and the sleeping conditions such as REM sleep and non-REM sleep, based on the values in the pulse wave interval data. The pulse wave interval data is calculated based on the pulse waves obtained by the pulse wave sensor 100.

The movement amount data is calculated based on the acceleration data obtained by the acceleration sensor 101. More specifically, a difference between two pieces of acceleration data that have been detected at two mutually different times is calculated as the movement amount data.

The memory 104 stores therein the user state data obtained by the user state recognizing unit 102 in correspondence with a measured time. After being transmitted to the information terminal 20, the data stored in the memory 104 is deleted from the memory 104 by the controlling unit 112, which is described later.

According to the first embodiment, the biological information is no longer necessary after the user state data is obtained. Thus, the memory 104 stores therein only the user state data. However, if the biological information is also necessary, the memory 104 may store therein the biological information measured by the pulse wave sensor 100 and the acceleration sensor 101, in correspondence with measured times.

The communication state recognizing unit 106 recognizes a current communication state. More specifically, the communication state recognizing unit 106 recognizes, as the communication state, an elapsed period of time since the immediately preceding time when the biological-information measuring apparatus 10 communicated with the information terminal 20, a current position of the biological-information measuring apparatus 10 derived from information obtained by, for example, a Global Positioning System (GPS) sensor or a sensor that measures a radio field intensity (not shown), and a communication state indicating whether the biological-information measuring apparatus 10 is in a state of being able to communicate with the information terminal 20.

The transmission timing judging unit 108 determines a communication timing at which the biological-information measuring apparatus should communicate with the information terminal 20, according to the communication state recognized by the communication state recognizing unit 106. The communicating unit 110 performs data transmission between the biological-information measuring apparatus 10 and the information terminal 20. The controlling unit 112 controls over the entire functions.

The information terminal 20 is, specifically, a mobile phone. As other examples, the information terminal 20 may be a Personal Digital Assistant (PDA), a portable game machine, or an exclusive-use terminal. The information terminal 20 is not limited to the example described in the exemplary embodiments. The information terminal 20 may be any kind of device as long as it has a communicating function to communicate with the biological-information measuring apparatus 10, a display unit used for providing information to the user, an operating unit that can be operated by the user, as well as a calculating function and is of a portable size. The biological-information measuring apparatus 10 wirelessly communicates with the information terminal 20. For example, the biological-information measuring apparatus 10 communicates with the information terminal 20, using Bluetooth.

The information terminal 20 includes a communicating unit 200, a detail analysis unit 202, a data accumulating unit 204, an operation displaying unit 206, a call communication unit 200, and a controlling unit 210. The communicating unit 200 communicates with the biological-information measuring apparatus 10.

The detail analysis unit 202 performs an analysis further in detail, based on the data received from the biological-information measuring apparatus 10. The process performed by the detail analysis unit 202 requires a larger amount of computation than the process performed by the user state recognizing unit 102 included in the biological-information measuring apparatus 10.

For example, as for the pulse wave interval data, the detail analysis unit 202 performs a Fast Fourier Transformation (FFT) on a predetermined range of the data (for example, a portion of the data corresponding to one minute). As a result, the detail analysis unit 202 calculates a Low Frequency (LF) component (i.e., a sympathetic nerve indicator) that is a component near 0.1 hertz (Hz) and a High Frequency (HF) component (i.e., a parasympathetic nerve indicator) that is a component near 0.3 Hz. The detail analysis unit 202 analyzes the state of autonomic nerves, based on these indicators. Further, the detail analysis unit 202 conjectures sleeping conditions based on a combination of, for example, the state of autonomic nerves and the movement amount derived from the acceleration data.

The data accumulating unit 204 accumulates therein the user state data obtained from the biological-information measuring apparatus 10 and detailed state data obtained by the detail analysis unit 202. When only the detailed state data that has been obtained based on the user state data is necessary, but the user state data itself is not necessary, the data accumulating unit 204 does not need to accumulate therein the user state data. In other words, an arrangement is acceptable in which the data accumulating unit 204 accumulates therein only one of the user state data and the detailed state data. The operation displaying unit 206 receives an operation instruction from the user and displays the measurement result according to the operation instruction. The call communication unit 208 performs a process related to the telephone.

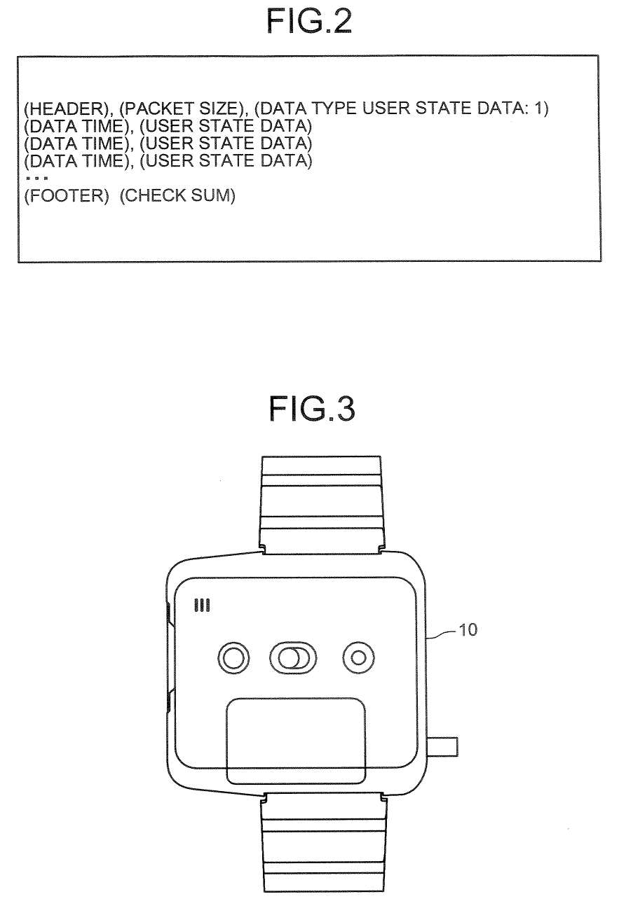

As shown in FIG. 2, each of pieces of user state data is stored in correspondence with a data time at which a piece of biological information corresponding to the piece of user state data has been recognized.

As shown in FIG. 3, the biological-information measuring apparatus 10 is an apparatus in the form of a wrist watch. The biological-information measuring apparatus 10 is wearable on the body of a user while measuring the biological information. The biological-information measuring apparatus 10 is not limited to the example described in the exemplary embodiments and may be in any form as long as it is wearable on the body of a user.

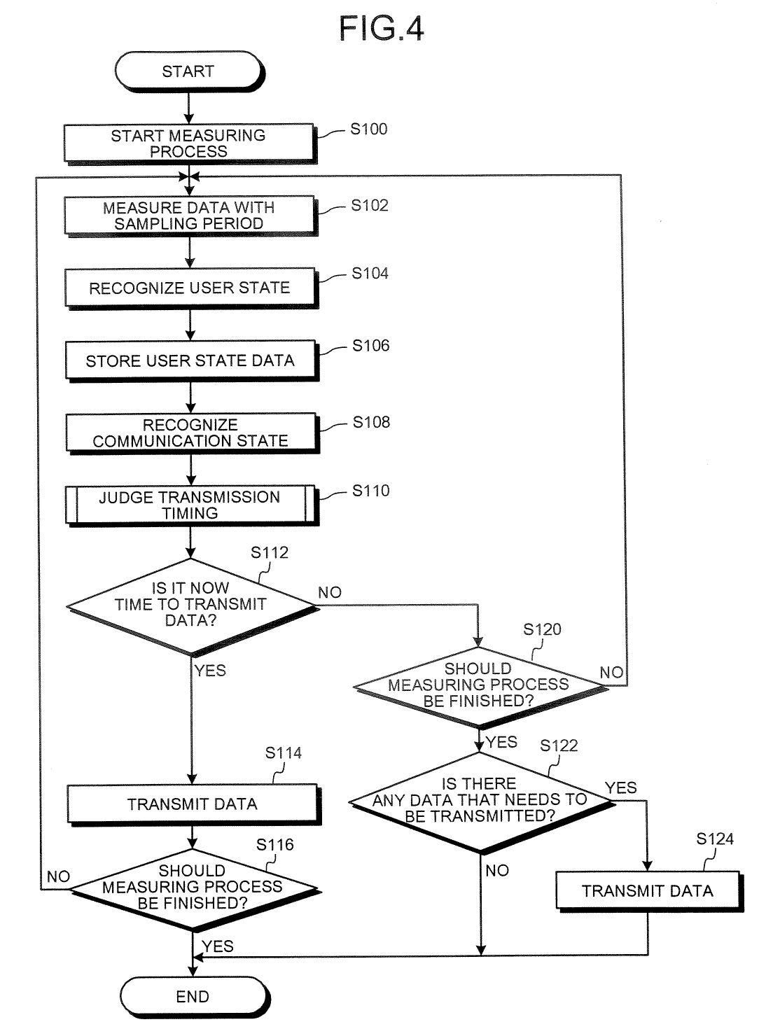

As shown in FIG. 4, when a user turns on the electric power of the biological-information measuring apparatus 10, the pulse wave sensor 100 and the acceleration sensor 101 start measuring biological information (step S100). The pulse wave sensor 100 and the acceleration sensor 101 continue to measure the biological information with a predetermined sampling period (step S102). As described above, when the electric power is turned on, the measuring process is automatically started. Alternatively, another arrangement is acceptable in which a measuring start button is provided so that the measuring process is started when the button is pushed.

Next, the user state is recognized based on the measured information obtained by the pulse wave sensor 100 and the acceleration sensor 101 (step S104). As a result, the pulse wave interval data and the movement amount data are obtained as the user state data.

As explained above, the biological-information measuring apparatus 10 performs only relatively simple processes. Processes that require a computational cost is performed by the information terminal 20. As a result, it is possible to reduce the electric power consumption of the biological-information measuring apparatus 10. When it is not necessary to reduce the electric power consumption of the biological-information measuring apparatus 10, another arrangement is acceptable in which the biological-information measuring apparatus 10 performs processes that require a higher computational cost.

Subsequently, the user state data obtained by the user state recognizing unit 102 is stored into the memory 104 (step S106). After that, the communication state recognizing unit 106 recognizes the communication state (step S108). The transmission timing judging unit 108 judges whether it is now a time to transmit the information stored in the memory 104 to the information terminal 20, based on the communication state (step S110).

When it has been judged that it is now a time to transmit the data (step S112: Yes), a connection between the biological-information measuring apparatus 10 and the information terminal 20 is established, and the data stored in the memory 104 is transmitted to the information terminal 20 (step S114). In the present example, it is assumed that the pieces of data as shown in FIG. 2 are transmitted in a sequential manner. The order in which the pieces of data are transmitted is not relevant.

As explained later, the memory 104 also stores therein data that has previously failed to be transmitted. At step S114, all of the data that is stored in the memory 104, including the data that has previously failed to be transmitted, is transmitted to the information terminal 20. With this arrangement, it is possible to transmit the data to the information terminal 20 without fail.

When the transmission of the data has been finished, the connection between the biological-information measuring apparatus 10 and the information terminal 20 is disconnected. Also, the data that has finished to be transmitted is deleted from the memory 104. When an instruction indicating that the measuring process should be finished is issued (step S116: Yes), the process performed by the biological-information measuring apparatus 10 is completed. When no instruction indicating that the measuring process should be finished is issued (step S116: No), the process returns to step S102 so that the pulse wave sensor 100 and the acceleration sensor 101 measure biological information.

It is assumed that the instruction indicating that the measuring process should be finished is issued when, for example, the electric power of the biological-information measuring apparatus 10 is turned off. Alternatively, another arrangement is acceptable in which the biological-information measuring apparatus includes a measuring finish button so that an instruction indicating that the measuring process should be finished is issued when the button is pushed.

As a result of the process performed at step S110, when it has been judged that it is not yet a time to transmit the data (step S112: No), and also an instruction indicating that the measuring process should be finished is issued (step S120: Yes), while some data that needs to be transmitted is stored in the memory 104 (step S122: Yes), it is judged whether it is now a time to transmit the data in the same manner as in the process performed at step S110. After it is confirmed that it is a time to transmit the data, and when the data has eventually been transmitted (step S124), the process performed by the biological-information measuring apparatus 10 is completed.

If the biological-information measuring apparatus 10 is in a state of being unable to communicate with the information terminal 20, the data remains to be stored in the memory 104 if the memory 104 is a non-volatile memory, so that the data is transmitted when the electric power is turned on again later. The data is discarded if the memory 104 is a volatile memory.

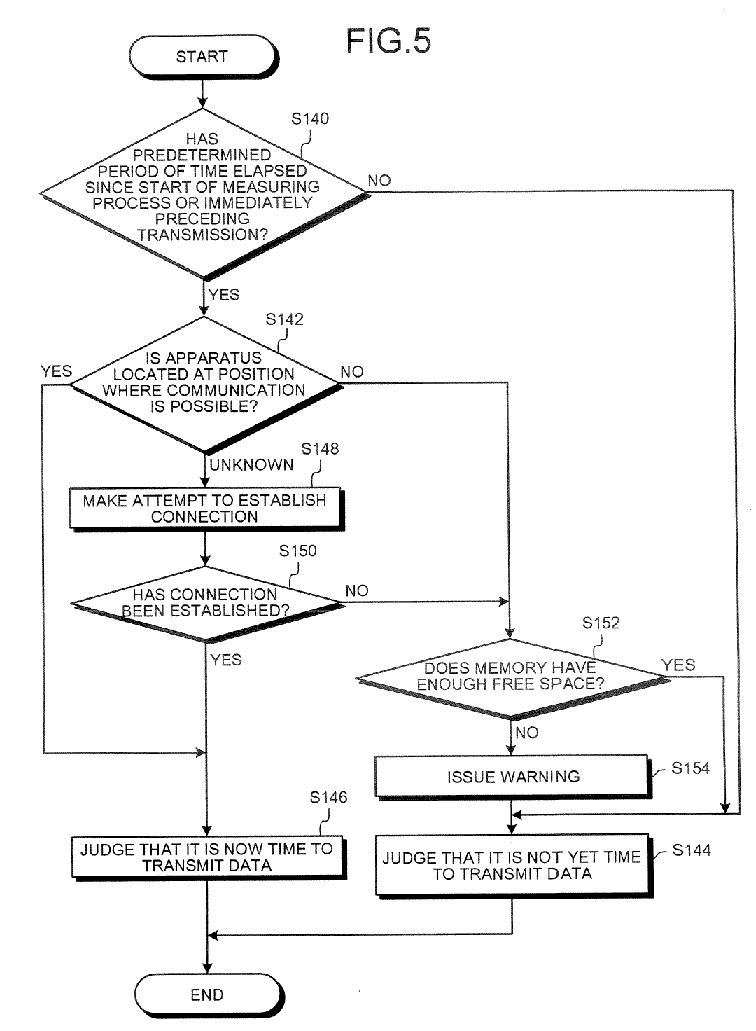

As shown in FIG. 5, in the transmission timing judging process (step S110) explained with reference to FIG. 4, the transmission timing judging unit 108 checks to see if a predetermined period of time has elapsed since the start (step S100) of the measuring process of the data. The predetermined period of time may be, for example, one minute. When the transmission has already been started, the transmission timing judging unit 108 checks to see if a predetermined period of time has elapsed since an immediately preceding transmission. The predetermined period of time used for judging the elapsed time since the start of the data measuring process may have the same length as, or may have a different length from, the predetermined period of time used for judging the elapsed time since the immediately preceding transmission.

When the predetermined period of time has elapsed since the start of the measuring process or since the immediately preceding transmission (step S140: Yes), the process proceeds to step S142. Conversely, when the predetermined period of time has not elapsed (step S140: No), it is judged that it is not yet a time to transmit the data (Step S144).

After that, it is judged whether the biological-information measuring apparatus 10 is located at a position where the biological-information measuring apparatus 10 is able to communicate with the information terminal 20. More specifically, when the biological-information measuring apparatus 10 was able to communicate with the information terminal 20 the immediately preceding time, and also if there has been no change in the acceleration data thereafter that suggests any movement of the biological-information measuring apparatus 10, it is considered that the biological-information measuring apparatus 10 is still located at a position where the biological-information measuring apparatus 10 is able to communicate with the information terminal 20. Thus, it is judged that the communication is possible. For example, a threshold value for the acceleration data may be set, so that it is judged that there has been no change in the acceleration data that suggests any movement of the biological-information measuring apparatus 10, if no acceleration data that is equal to or higher than the threshold value is obtained.

When the biological-information measuring apparatus 10 was not able to communicate with the information terminal 30 the immediately preceding time, and also if there has been no change in the acceleration data thereafter that suggests any movement of the biological-information measuring apparatus 10, it is considered that the biological-information measuring apparatus 10 is still located at a position where the biological-information measuring apparatus 10 is unable to communicate with the information terminal 20. Thus, it is judged that the communication is not possible.

When the process at this step (i.e., step S142) is performed for the first time after the start of the measuring process of the data, it is not possible to judge whether the biological-information measuring apparatus 10 is located at a position where the biological-information measuring apparatus 10 is to able to communicate with the information terminal 20. Thus, in such a situation, it is judged that the communicability is unknown. Also, regardless of whether the biological-information measuring apparatus 10 was able to communicate with the information terminal 20 the immediately preceding time, when there has been a change in the acceleration data that suggests movement of the biological-information measuring apparatus 10 since the last communication, it is not possible to judge whether the current positional relationship allows the biological-information measuring apparatus 10 to communicate with the information terminal 20. Thus, in this situation also, it is judged that the communicability is unknown.

According to the rules defined above, when it has been judged that the biological-information measuring apparatus 10 is located at a position where the biological-information measuring apparatus 10 is able to communicate with the information terminal 20 (step S142: Yes), it is judged that it is now a time to transmit the data (step S146).

When it has been judged that it is unknown whether the biological-information measuring apparatus 10 is located at a position where the biological-information measuring apparatus 10 is able to connect to the information terminal 20 (step S142: unknown), the communicating unit 110 makes an attempt to establish a connection to the information terminal 20 (step S148). When a connection has been established (step S150: Yes), it is judged that it is now a time to transmit the data (step S146).

When no connection has been established (step S150: No), it is checked to see if the memory 104 has enough free space in which the data to be transmitted can be stored. When the memory 104 does not have enough free space (step S152: No), a warning is issued (step S154). As the warning, a Light-Emitting Diode (LED) may be turned on or a warning sound may be made. With this arrangement, it is possible to prompt the user to assure a good communication state. After that, it is judged that it is not yet a time to transmit the data (step S144).

On the other hand, when the memory 104 has enough free space (step S152: Yes), no warning is issued, and it is judged that it is not yet a time to transmit the data (step S144).

When it has been judged at step S142 that the biological-information measuring apparatus 10 is not located at a position where the biological-information measuring apparatus 10 is able to communicate with the information terminal 20 (step S142: No), it is judged that it is not yet a time to transmit the data, and it is checked to see if the memory 104 has enough free space (steps S152, S154, and S144). Thus, the process at step S110 is completed.

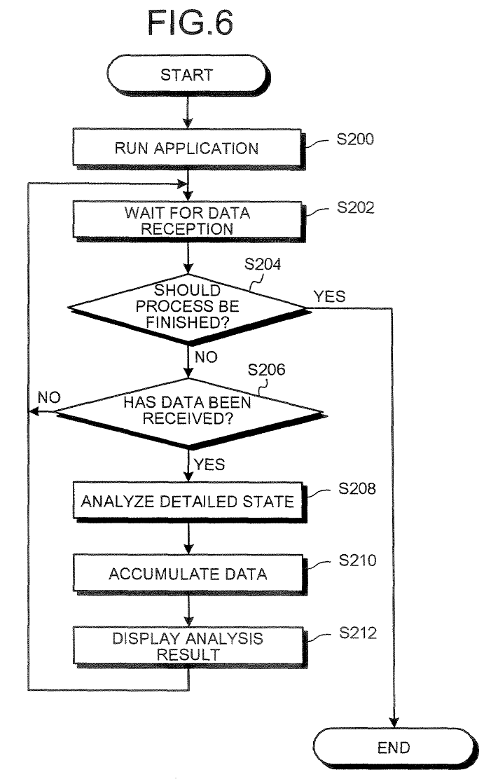

As shown in FIG. 6, the information terminal 20 runs an application that receives, browses, and manages data (step S200). These processes at step S200 do not need to be performed if the information terminal 20 is an exclusive-use terminal. After the application starts running, the application is in a state of waiting for data reception (step S202). In other words, the application stands by in such a state that it is possible to respond any time to a connection request from the biological-information measuring apparatus 10.

When data is received while the application is in the state of waiting for data reception (step S204: No; step S206: Yes), the detail analysis unit 202 performs an analysis further in detail, based on the received data (step S208). Subsequently, detailed state data obtained as a result of the analysis performed by the detail analysis unit 202 and the user state data are accumulated in the data accumulating unit 204 (step S210). After that, an analysis result at this time is displayed on the operation displaying unit 206 (step S212), and the process returns to step S202. The process described above is repeated until the user enters an input indicating that the process should be finished (step S204: Yes), and the process is completed when the user has entered an input indicating that the process should be finished.

As explained above, the data is transmitted after it has been confirmed that the biological-information measuring apparatus 10 is located at a position where the biological-information measuring apparatus 10 is able to communicate with the information terminal 20. When it has been judged that the biological-information measuring apparatus 10 is not located at a position where the biological-information measuring apparatus 10 is able to communicate with the information terminal 20, the data is transmitted later. Thus, it is possible to transmit the data without fail. Also, there is no need to maintain the state in which the biological-information measuring apparatus 10 is able to communicate with the information terminal 20 at all times, therefore, it is possible to reduce the electric power consumption required by the communication. Furthermore, it is possible to transmit the data automatically every time the predetermined period of time has elapsed, without receiving any instruction from the user.

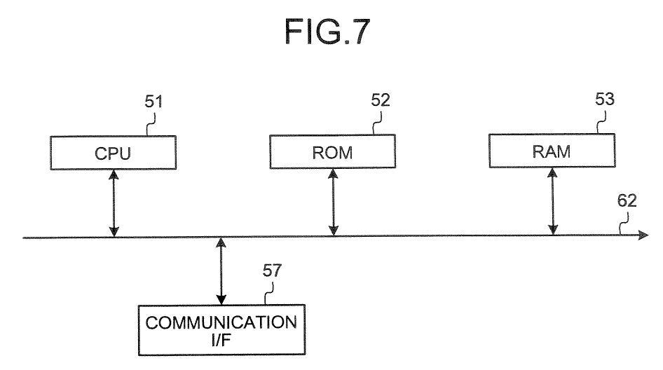

As shown in FIG. 7, the biological-information measuring apparatus 10 includes, as its hardware configuration, a Read-Only Memory (ROM) 52 that stores therein, for example, a biological information measuring program for executing the biological information measuring process by the biological-information measuring apparatus 10, a Central Processing Unit (CPU) 51 that controls the constituent elements of the biological-information measuring apparatus 10 according to the program stored in the ROM 52, a Random Access Memory (RAM) 53 that stores therein various types of data that are required in the control of the biological-information measuring apparatus 10, a communication interface (I/F) 57 that establishes a connection to a network and performs communication, and a bus 62 that connects these constituent elements to one another.

The biological information measuring program mentioned above that is used by the biological-information measuring apparatus 10 may be provided as being recorded on a computer-readable recording medium such as a Compact Disk Read Only Memory (CD-ROM), a floppy® disk (FD) or a Digital Versatile Disk (DVD), in a file that is in an installable format or in an executable format.

In such a situation, the biological information measuring program is loaded into a main storage device when being read from the recording medium and executed by the biological-information measuring apparatus 10, so that the constituent elements that are explained in the description of the software configuration are generated in the main storage device.

Further, another arrangement is acceptable in which the biological information measuring program according to the first embodiment is stored in a computer connected to a network like the Internet so that the biological information measuring program is provided as being downloaded via the network. The hardware configuration of the information terminal 20 is the same as the hardware configuration of the biological-information measuring apparatus 10.

The present invention has been explained so far according to the first embodiment; however, it is possible to modify or improve the first embodiment in various ways.

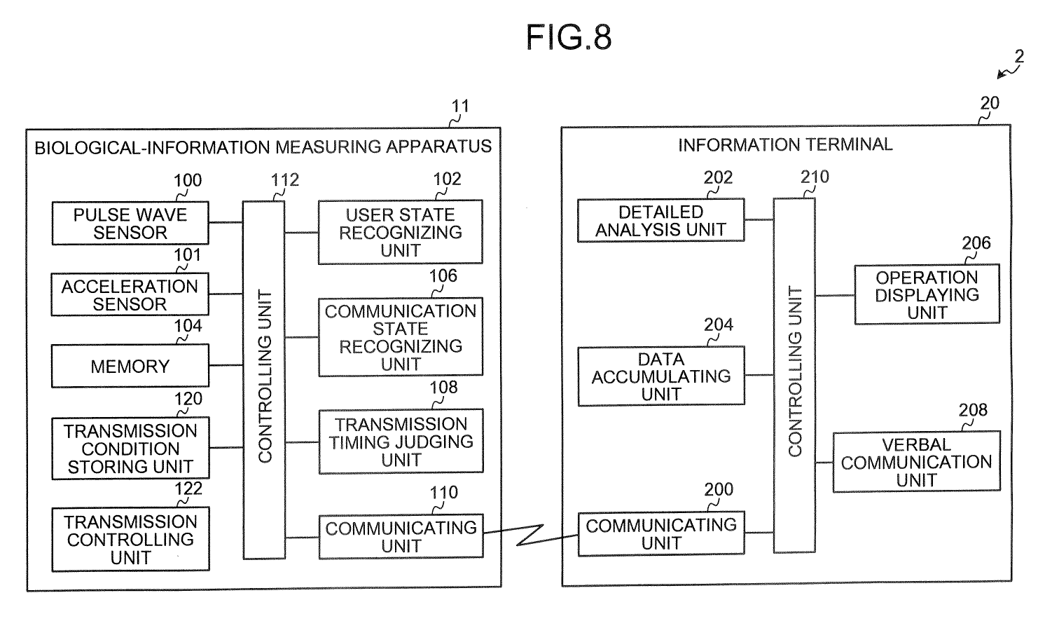

As shown in FIG. 8, in a biological information measuring system 2 according to a second embodiment, a biological-information measuring apparatus 11 transmits biological information to the information terminal 20 at a predetermined time.

To reduce the amount of data to be transmitted, it is desirable to transmit only the user state data that is needed by the information terminal 20, without transmitting the biological information on which the user state data is based. However, those who analyze the data such as service providers and an administrator of the biological information measuring system 2 may wish to obtain the biological information for the purpose of improving the precision level of the state recognition by continuously collecting and studying raw data. For example, they may wish to obtain raw data when a signal-to-noise (S/N) ratio of signals is low, or when signals in data obtained while a user is asleep are disrupted because the user frequently rolls over in his/her sleep.

Thus, there is a situation where the biological information needs to be continuously transmitted so that the biological information can be used for the management purposes. However, in such a situation, the amount of data to be transmitted increases, and the electric power consumption of the biological-information measuring apparatus 11 also increases. To cope with this situation, according to the second embodiment, among the biological information that has been measured, only a portion that satisfies a predetermined condition, such as having a low S/N ratio, is transmitted to the information terminal 20, in addition to the user state data.

The biological-information measuring apparatus 11 included in the biological information measuring system 2 according to the second embodiment further includes a transmission condition storing unit 120 and a transmission controlling unit 122, in addition to the functional configuration included in the biological information measuring system 1 according to the first embodiment. The transmission condition storing unit 120 stores therein a transmission condition that is specified in advance. The transmission condition is a condition under which some biological information is also transmitted in addition to the user state data. In other words, when the transmission condition is satisfied, some biological information is also transmitted in addition to the user state data. More specifically, the transmission condition may be, for example, “a portion in which the amplitude of the pulse wave is equal to or smaller than a defined amplitude value” or “a portion in which the movement amount is equal to or larger than a defined movement amount value”. It is assumed that specific values are given as the defined amplitude value and the defined movement amount value. Any arbitrary values can be set as the defined amplitude value and the defined movement amount value.

The transmission condition may be the same for all users. Alternatively, the transmission condition may vary for each user. Further alternatively, another arrangement is acceptable in which each user is able to specify a transmission condition by performing an operation.

The transmission controlling unit 122 monitors whether the transmission condition is satisfied. More specifically, the transmission controlling unit 122 monitors the pulse wave amplitude obtained by the pulse wave sensor 100 and the movement amount obtained by the acceleration sensor 101. When having judged that the transmission condition is satisfied, the transmission controlling unit 122 instructs the communicating unit 110 to transmit, to the information terminal 20, not only the user state data, but also a corresponding portion of the biological information that satisfies the transmission condition. To summarize, the transmission controlling unit 122 determines what data should be transmitted to the information terminal 20.

The transmission controlling unit 122 according to the second embodiment judges whether any biological information should be transmitted. Alternatively, the transmission controlling unit 122 may be configured so as to judge whether the user state data should be transmitted.

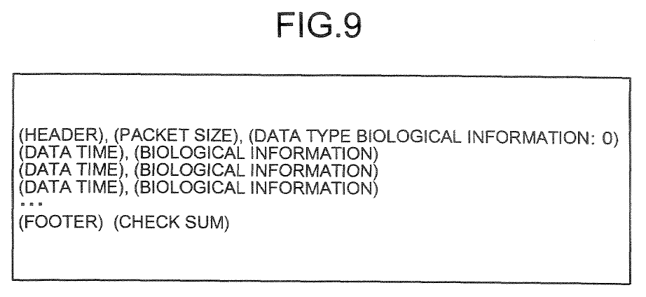

As shown in FIG. 9, each of pieces of biological information is stored in correspondence with a data time at which the piece of biological information has been measured.

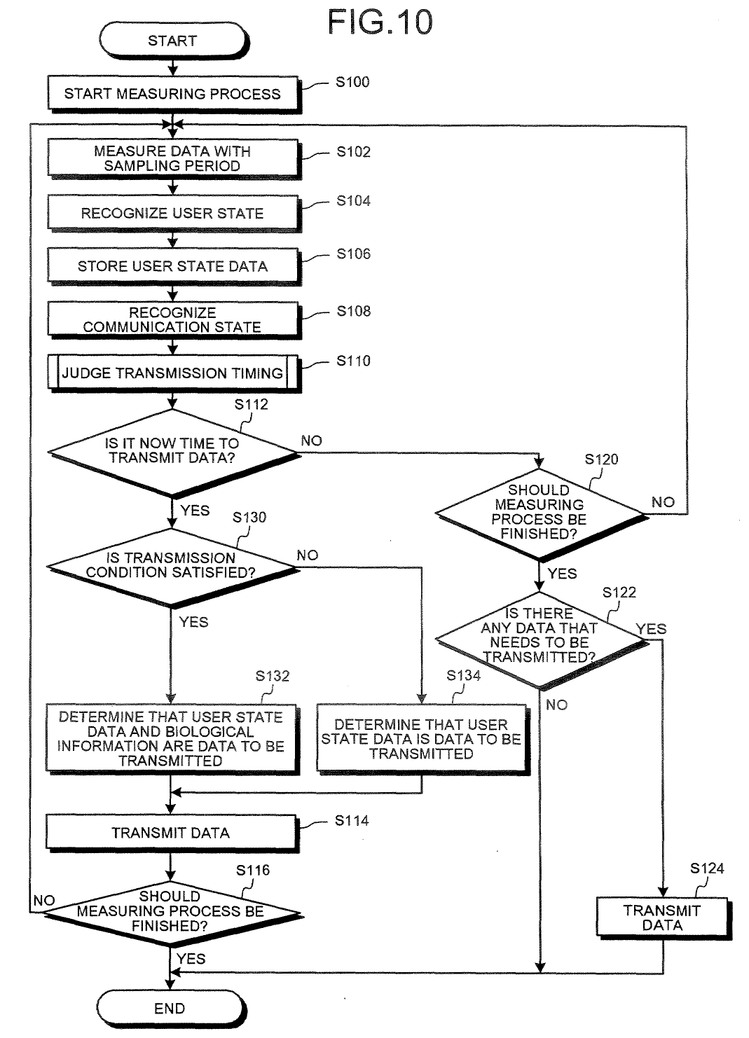

As shown in FIG. 10, in the biological-information measuring apparatus 11 according to the second embodiment, when it has been judged that it is now a time to transmit the data (step S112: Yes), the transmission controlling unit 122 judges whether the transmission condition stored in the transmission condition storing unit 120 is satisfied. When the transmission condition is satisfied (step S130: Yes), the transmission controlling unit 122 determines that biological information as well as the user state data are the data to be transmitted (step S132).

On the other hand, when the transmission condition is not satisfied (step S130: No), the transmission controlling unit 122 determines that only the user state data is the data to be transmitted (step S134). The communicating unit 110 then transmits, to the information terminal 20, the data that has been determined by the transmission controlling unit 122 as the data to be transmitted (Step S114).

Also, immediately before the data transmission process (step S124) is performed, the transmission controlling unit 122 determines, in the same fashion, what data should be transmitted, based on whether the transmission condition is satisfied. After that, during the data transmission process (step S124), the communicating unit 110 transmits, to the information terminal 20, the data that has been determined by the transmission controlling unit 122 as the data to be transmitted.

Other configurations and processes of the biological information measuring system 2 according to the second embodiment are the same as the configurations and the processes of the biological information measuring system 1 according to the first embodiment.

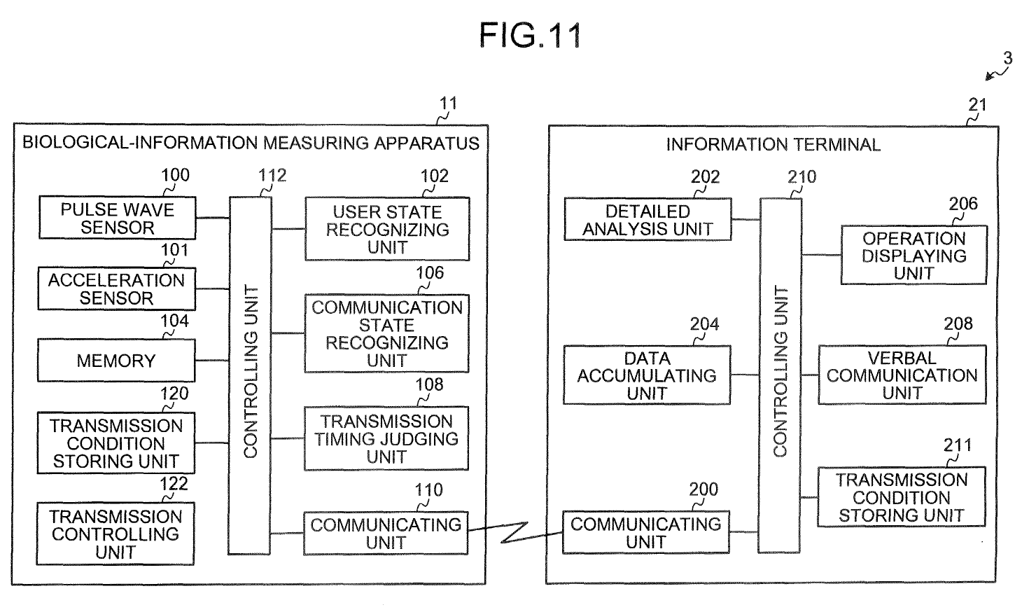

As shown in FIG. 11, in a biological information measuring system 3 according to a first modification example of the second embodiment, an information terminal 21 includes a transmission condition storing unit 211 that stores therein a transmission condition. Other functional configurations are the same as the functional configurations of the biological information measuring system 2 according to the second embodiment.

A service provider who uses the biological information measuring system 3 specifies, in advance, a transmission condition into the transmission condition storing unit 211 included in the information terminal 21. Then, the communicating unit 200 included in the information terminal 21 transmits the transmission condition stored in the transmission condition storing unit 211 to the biological-information measuring apparatus 11. The transmission condition storing unit 120 included in the biological-information measuring apparatus 11 stores therein the transmission condition received from the information terminal 21.

Further, another arrangement is acceptable in which the service provider or the like specifies a desired transmission condition into the biological-information measuring apparatus 11. When the biological-information measuring apparatus 11 is designed to be smaller than the information terminal 21, if the biological-information measuring apparatus 11 is configured to have many functions, the operability of the biological-information measuring apparatus 11 becomes low, and a heavy burden is put on the operator. Thus, by having the arrangement that allows the operator to specify the transmission condition by using the information terminal 21, it is possible to reduce the burden on the operator. In such a situation, when the transmission condition is changed according to an instruction from a user, the new transmission condition obtained after the change is transmitted from the information terminal 21 to the biological-information measuring apparatus 11. With this arrangement, even if the transmission condition has been changed, it is possible to automatically perform a process that reflects the new transmission condition obtained after the change.

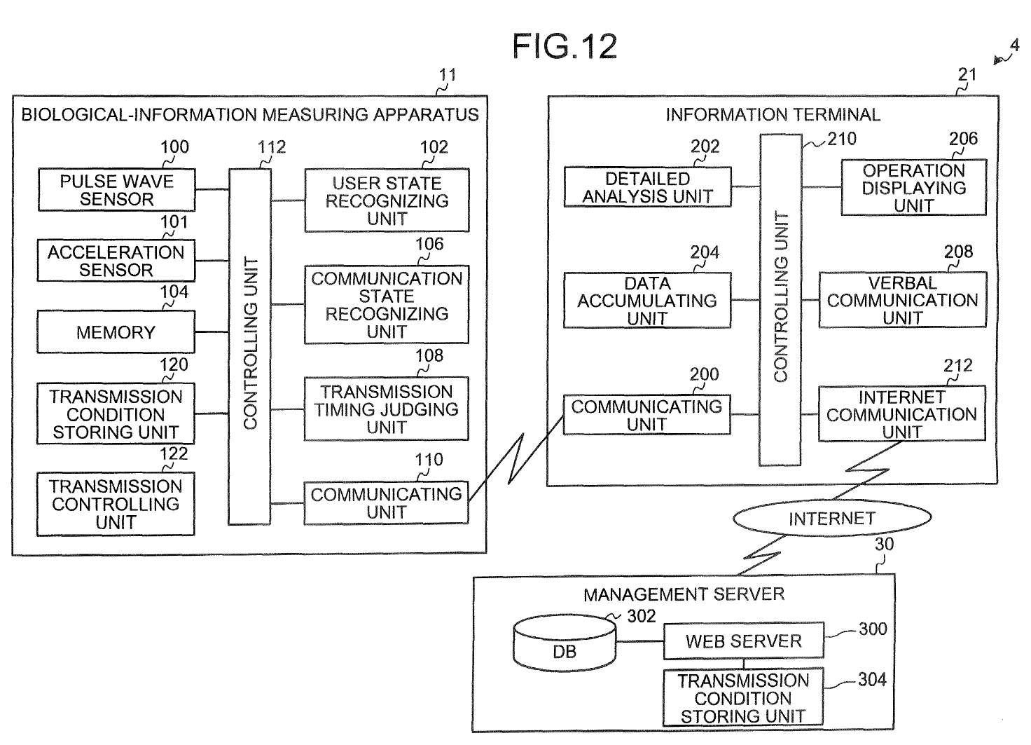

As shown in FIG. 12, a biological information measuring system 4 according to a second modification example of the second embodiment further includes a management server 30. In this system, a service provider who uses the biological information measuring system 4 specifies a transmission condition into the management server 30 in advance.

The management server 30 includes a Web server 300 that manages information transmission and the like, a database (DB) 302 that stores therein information to be provided, and a transmission condition storing unit 304 that stores therein a transmission condition. The transmission condition that has been specified into the transmission condition storing unit 304 is transmitted, via the Internet, to an Internet communication unit 212 included in the information terminal 21. Like according to the first modification example, the communicating unit 200 included in the information terminal 21 transmits the transmission condition that has been received via the internet communication unit 212 to the biological-information measuring apparatus 11. Further, another arrangement is acceptable in which the service provider is able to change the transmission condition stored in the transmission condition storing unit 304.

Furthermore, yet another arrangement is acceptable in which the management server 30 stores therein user IDs each identifying a user, in correspondence with their respective transmission conditions. With this arrangement, it is possible to specify an individual transmission condition for each of the users.

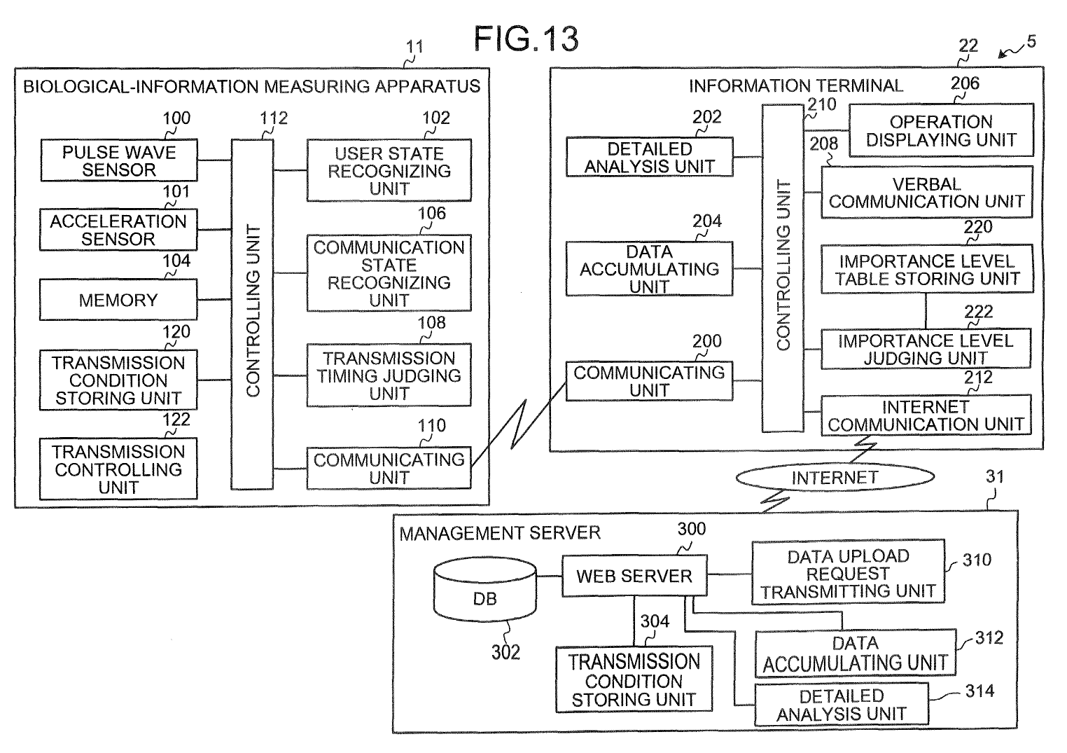

As shown in FIG. 13, a biological information measuring system 5 according to a third embodiment further includes a management server 31 in addition to the biological-information measuring apparatus 11 and an information terminal 22. Of a result of a detailed analysis, the information terminal 22 uploads only the data that is desired by the service provider to the management server 31.

The biological information measuring system 5 according to the third embodiment is similar to the biological information measuring system 4 according to the second modification example of the second embodiment; however, the information terminal 22 further includes an importance-level-table storing unit 220 and an importance level judging unit 222. The management server 31 further includes a data-upload-request transmitting unit 310, a data accumulating unit 312, and a detail analysis unit 314.

The data-upload-request transmitting unit 310 transmits a data upload request to the information terminal 22. The data upload request is information requesting that the data stored in the information terminal 22 should be uploaded onto the management server 31. The data upload request includes a measurement time period of the data that is requested to be uploaded and a level of importance. The level of importance is information that identifies the data that is requested to be uploaded. The data upload request is written as, for example, “20060101-20060107; the level of importance—high”. It means that such a portion of the data that has been measured from Jan. 1, 2006 to Jan. 7, 2006, that corresponds to a high level of importance is requested to be uploaded.

The data accumulating unit 312 accumulates therein the data that is received in response to the data upload request that has been transmitted by the data-upload-request transmitting unit 310. The received data is biological information and/or detailed analysis data.

The detail analysis unit 314 performs an analysis on the biological information and the detailed analysis data. It is desirable to have an arrangement in which the analysis performed by the detail analysis unit 314 requires a larger amount of computation than the analysis performed by the detail analysis unit 202 included in the information terminal 22. By having this arrangement in which the management server 31 performs the analysis that requires a relatively larger amount of computation, it is possible to reduce the data processing amounts of the biological-information measuring apparatus 11 and the information terminal 22.

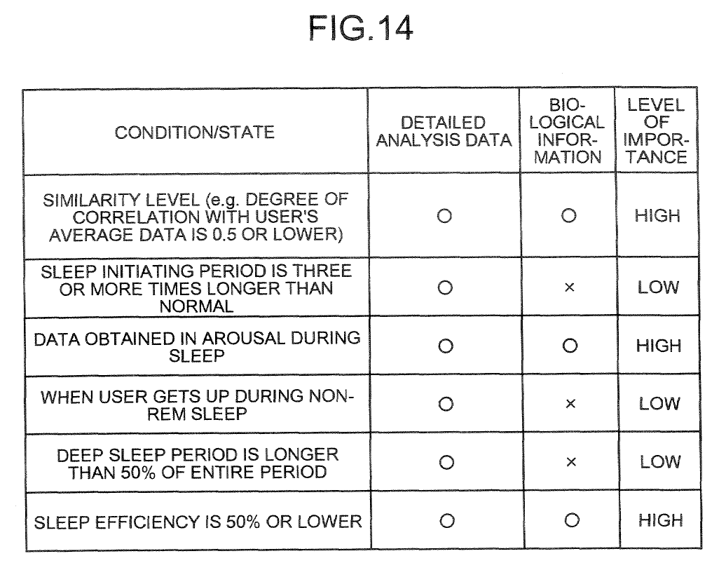

The importance-level-table storing unit 220 stores therein an importance level table. The importance level table shows various conditions in correspondence with corresponding levels of importance. As shown in FIG. 14, the importance level table stored in the importance-level-table storing unit 220 shows conditions and states in correspondence with the levels of importance. Further, types of data that are to be transmitted are shown in correspondence with the levels of importance. For example, when the level of importance is low, it means that only the detailed analysis data is to be transmitted. When the level of importance is high, it means that both the biological information and the detailed analysis data are to be transmitted.

Alternatively, another arrangement is acceptable in which, when the level of importance is high, only the biological information is to be transmitted. Further alternatively, it is acceptable to have a larger number of levels of importance than the two levels of importance, namely, high and low. As explained above, the number of levels of importance and the number of types of data that are to be transmitted in correspondence with each of the levels of importance are not limited to the example described in the exemplary embodiments.

In the example shown in FIG. 14, for example, a detailed analysis is performed by judging sleeping conditions of a user. When the sleeping condition shows a low degree of correlation (e.g. 0.5 or lower) with the user's average sleeping condition, in other words, when the similarity level is low, the level of importance is set to high. This is because a further analysis needs to be performed based on not only the detailed analysis data but also the biological information.

When a sleep initiating period (i.e., a period of time between the time when a user goes to bed and when he/she actually falls asleep) is three or more times longer than normal, an evaluation based on a result of a detailed analysis is sufficient, and no biological information is necessary. Thus, the level of importance is set to low. The sleep initiating period is measured by using the measuring start time as the time at which the user goes to bed.

When the user wakes up during sleep (i.e., arousal during sleep) or when the sleep efficiency is 50% or lower, the level of importance is set to high. The sleep efficiency is a ratio of the sleeping period of time to the period of time between the time when a user goes to bed and when the user gets out of bed. It is calculated as the ratio of the sleeping period of time to the period of time between a measuring start time and a measuring finish time.

When a user gets up during non-REM sleep and when the ratio of deep-sleep periods to the entire sleeping period is 50% or higher, the level of importance is set to low in each case.

It is preferable to set each of the levels of importance so that the lower the similarity level of a piece of data with respect to other pieces of data is, the higher the level of importance is. Also, it is preferable to set each of the levels of importance so that the higher the demand of the service provider for the data is, the higher the level of importance is. The conditions and the levels of importance may be set by a server administrator or the like, depending on the purpose for which the data is obtained.

Alternatively, another arrangement is acceptable in which the service provider or the like is able to configure the importance level table by using the information terminal 22. Further alternatively, yet another arrangement is acceptable in which the service provider or the like is able to configure the importance level table by using the management server 31. In this situation, the information terminal 22 receives the importance level table from the management server 31, so that the importance-level-table storing unit 220 stores therein the received importance level table.

By referring to the importance-level-table storing unit 220, the importance level judging unit 222 judges the level of importance of the data stored in the data accumulating unit 204. When having received a data upload request from the management server 31, the Internet communication unit 212 transmits, to the management server 31, a piece of data that satisfies the period of time and the level of importance written in the received data upload request.

When uploading of the data is left up to the initiative of the user or the like, it tends to be difficult to obtain a sufficient amount of data or a desired type of data. However, according to the arrangement described above, because the levels of importance are set in advance, it is possible to upload, on the server side, only the desired data from the information terminal 22 by simply specifying a level of importance. Further, because it is possible to suppress the target of the uploading process to the requisite minimum, it is possible to reduce the communication costs to the minimum level.

Other configurations and processes of the biological information measuring system 5 according to the third embodiment are the same as the configurations and the processes of the biological information measuring systems according to the other exemplary embodiments. The hardware configuration of the management server 31 is the same as the hardware configuration of the biological-information measuring apparatus 10 that is explained in the description of the first embodiment with reference to FIG. 7.

As a first modification example of the third embodiment, another arrangement is acceptable in which a data upload request is transmitted regularly. With this arrangement, it is possible to upload desired data regularly.

As a second modification example of the third embodiment, yet another arrangement is acceptable in which, when the management server 31 receives data from a plurality of information terminals 22, the same data upload request is transmitted each of all the information terminals 22 that communicate with the management server 31. Alternatively, mutually different data upload requests may be respectively transmitted to the information terminals 22 that communicate with the management server 31.

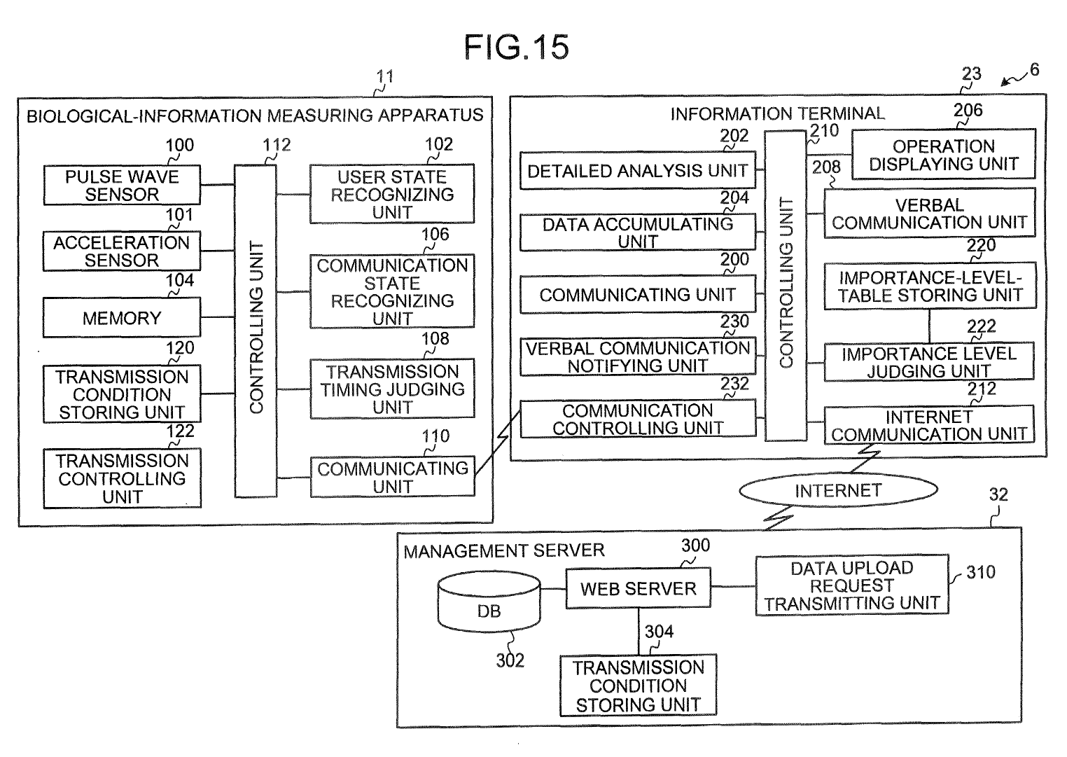

As shown in FIG. 15, a biological information measuring system 6 according to a fourth embodiment is similar to the biological information measuring system 5 according to the third embodiment; however, an information terminal 23 according to the fourth embodiment is a terminal such as a mobile phone that has a call communication function, although the information terminal 22 according to the third embodiment may be a mobile terminal or a PDA. Also, the information terminal 23 according to the fourth embodiment further includes a call communication notifying unit 230 and a communication controlling unit 232, in addition to the functional configuration of the information terminal 22 according to the third embodiment.

When the call communication unit 208 has received a call, the call communication notifying unit 230 instructs the communicating unit 200 to notify the biological-information measuring apparatus 11 that a call has been received. The communication controlling unit 232 disconnects the communication between the information terminal 23 and the biological-information measuring apparatus 11 while call communication is performed. Also, when the call communication has been finished, the communication controlling unit 232 re-connects the information terminal 23 to the biological-information measuring apparatus 11.

If the information terminal 23 is a mobile phone, when a call has been received and while call communication is performed, in principle, all the other functions besides the telephone function are suspended. To cope with this situation, by having the functional configuration described above, it is possible to switch between the process operations when a call has been received during a measuring process.

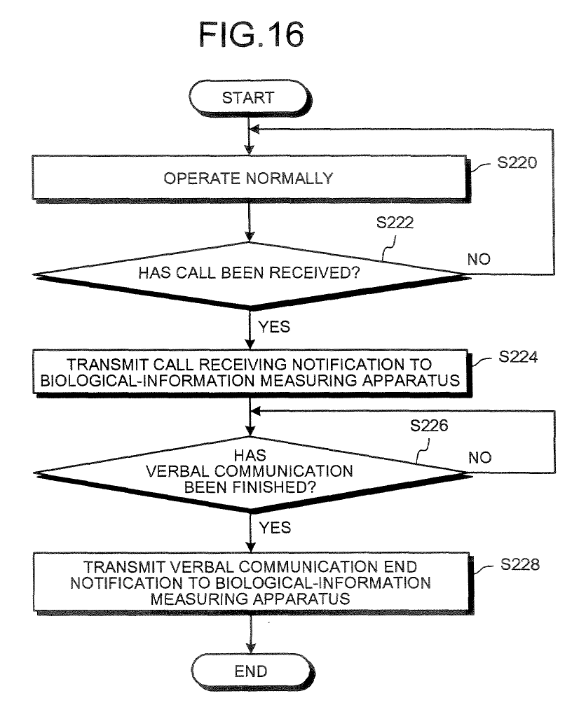

As shown in FIG. 16, during a call receiving process performed by the information terminal 23, if a call is received (step S222: Yes) while the normal process is performed (step S220), the communicating unit 200 transmits a call receiving notification to the biological-information measuring apparatus 11 according to an instruction from the call communication notifying unit 230 (step S224). Here, the normal process was explained in the description of the first embodiment with reference to FIG. 6. When the call communication has been finished (step S226: Yes), the communicating unit 200 transmits a call communication end notification to the biological-information measuring apparatus 11 according to an instruction from the call communication notifying unit 230 (step S228). Thus, the call receiving process is completed.

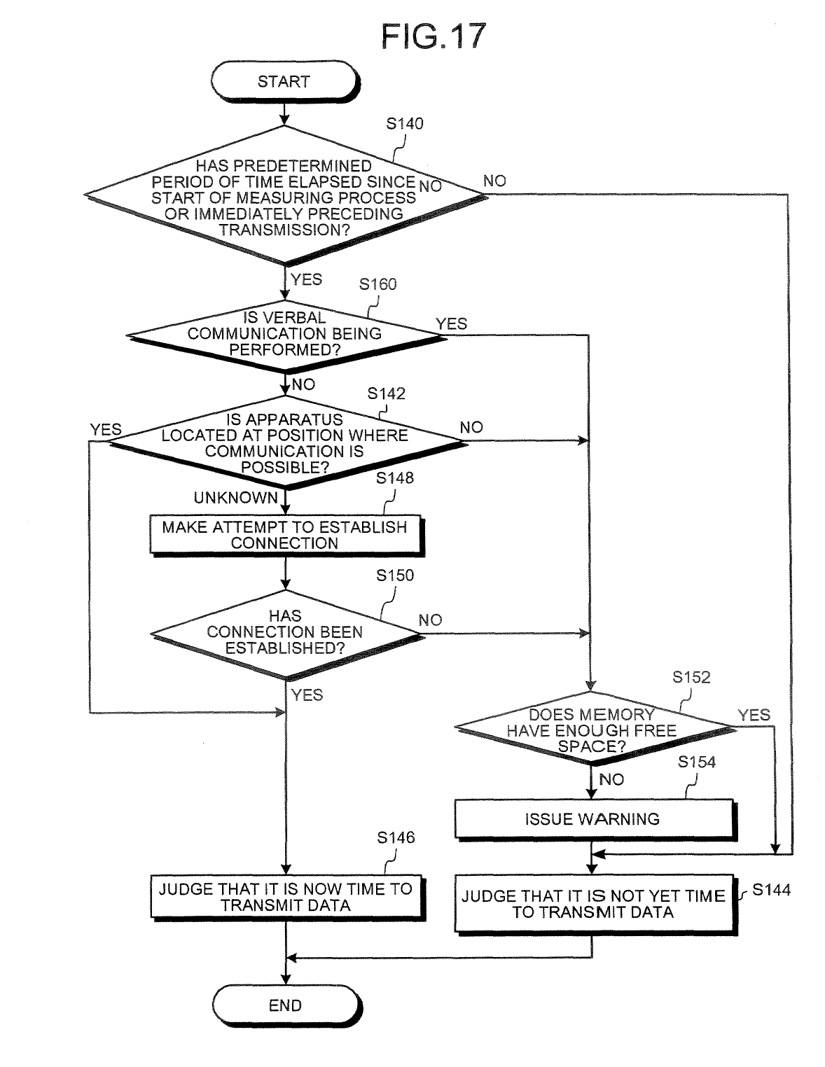

As shown in FIG. 17, during the transmission timing judging process (step S110), the biological-information measuring apparatus 11 considers that the information terminal 23 is performing call communication for the period of time between the time when the communicating unit 110 receives the call receiving notification and when the communicating unit 110 receives the call communication end notification. If the information terminal 23 is performing call communication (step S160: Yes), the process proceeds to step S152, and it is judged that it is not yet a time to transmit the data. Thus, the data is accumulated in the memory 104. In other words, the biological-information measuring apparatus 11 operates in a logger mode.

After a call communication end notification has been received, the biological-information measuring apparatus 11 recognizes that the information terminal 23 is not performing call communication (step S160: No), and the process proceeds to step S142. In other words, the biological-information measuring apparatus 11 returns to a normal mode from the logger mode.

As explained above, in the biological information measuring system 6 according to the fourth embodiment, if the information terminal 23 is performing call communication, and the biological-information measuring apparatus 11 is therefore not able to transmit the data to the information terminal 23, the data that needs to be transmitted is accumulated into the memory 104. The data that has been accumulated in the memory 104 is transmitted to the information terminal 23 after the call communication is finished. With this arrangement, it is possible to transmit the data at an appropriate time without fail.

Other configurations and processes of the biological information measuring system 6 according to the fourth embodiment are the same as the configurations and the processes of the biological information measuring system 5 according to the third embodiment.

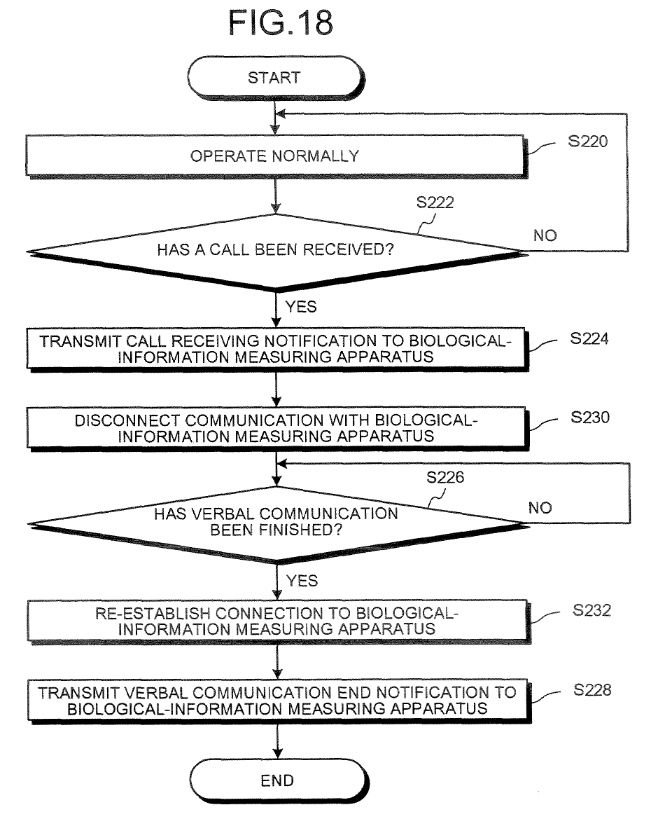

The call receiving process according to the fourth embodiment is applied in such a situation with, for example, Bluetooth, where the communication is disconnected when a call has been received. However, when a method by which the communication is not disconnected even if a call has been received is used, a call receiving process according to a first modification example of the fourth embodiment as shown in FIG. 18 is performed.

More specifically, when a call has been received (step S222: Yes), the communication between the biological-information measuring apparatus 11 and the information terminal 23 is disconnected (step S230), and the process proceeds to step S226. When the call communication has been finished (step S226: Yes), the information terminal 23 is re-connected to the biological-information measuring apparatus 11 (Step S232), and the process proceeds to step S228. In this situation, there is no need to transmit the call receiving notification or the call communication end notification.

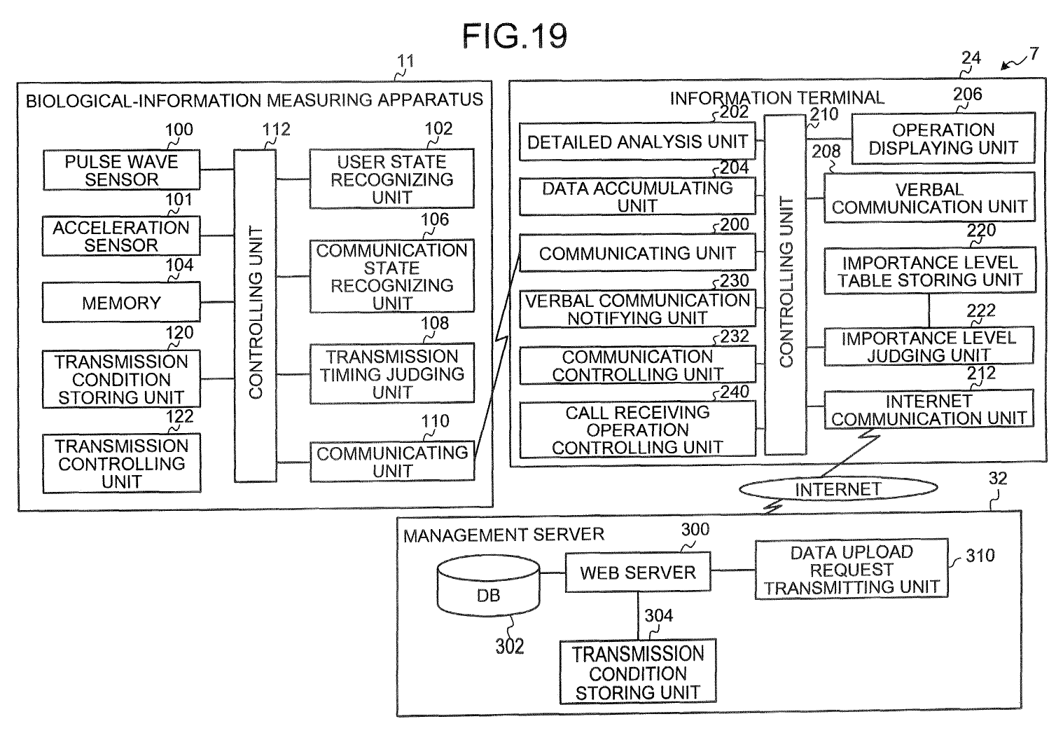

As shown in FIG. 19, a biological information measuring system 7 according to a fifth embodiment is further configured so that a call receiving mode of an information terminal 24 is changed depending on whether the user is awake or asleep. The biological information measuring system 7 according to the fifth embodiment further includes a call-receiving-operation controlling unit 240, in addition to the functional configuration of the information terminal 23 according to the fourth embodiment.

When the detail analysis unit 202 has detected that the user has fallen asleep, the call-receiving-operation controlling unit 240 sets the call receiving mode to a silent mode that uses, for example, a vibrator to notify that a call is being received. When the user gets up, the silent mode is cancelled. In other words, the call receiving mode is set to a normal mode in which a call receiving sound (i.e., a ring-tone) is made. With this arrangement, it is possible to automatically change the call receiving operation, according to the state of the user. Thus, the user does not need to set the call receiving mode. Further, the call-receiving-operation controlling unit 240 changes the call receiving mode depending on whether the user is awake or asleep.

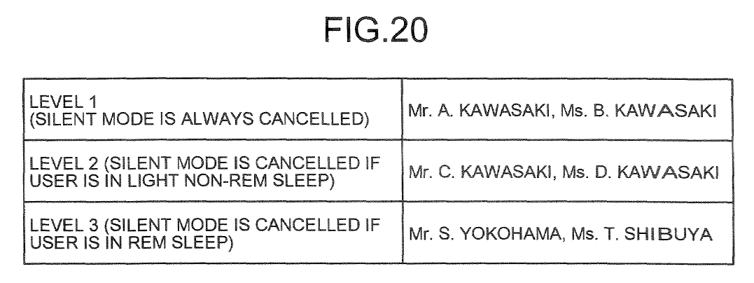

As shown in FIG. 20, a call receiving mode determination table stored in the call-receiving-operation controlling unit 240 stores therein conditions under each of which the call receiving mode is changed, in correspondence with call communication partners. The call-receiving-operation controlling unit 240 determines the call receiving operation by referring to the call receiving mode determination table. For example, at Level 1, the condition is set so that, when a call is received from any one of the specified call communication partners, the silent mode is always cancelled. At Level 2, the condition is set so that, when a call is received from any one of the specified call communication partners, the silent mode is cancelled if the user is in light non-REM sleep. At Level 2, the silent mode is cancelled also if the user is in REM sleep. The silent mode is not cancelled if the user is in deep non-REM sleep. At Level 3, the condition is set so that the silent mode is cancelled if the user is in REM sleep.

People who have a possibility of making an emergency call are registered at Level 1. Thus, when a call is received from any one of the call communication partners that are registered at Level 1, the silent mode is cancelled regardless of the sleeping condition of the user so that the user is able to answer the phone. People who have a low possibility making emergency calls, such as friends, are registered at Level 3. Thus, the calls are answered by an answering machine or the like. With this arrangement, it is possible to control the call receiving operation according to the judgment result regarding the sleeping condition of the user that is obtained by the information terminal 24.

Other configurations and operations of the biological information measuring system 7 according to the fifth embodiment are the same as the configurations and operations of the biological information measuring system 6 according to the fourth embodiment.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A biological information measuring system comprising:
a measuring apparatus that measures biological information of a user; and
an information terminal that manages the biological information measured by the measuring apparatus, wherein
the measuring apparatus includes:
a sensor that measures the biological information;
a first storing unit that stores a measurement result obtained by the sensor;
a communication state recognizing unit that recognizes a current communication state of communication between the measuring apparatus and the information terminal;
a determining unit that determines a transmission timing capable of transmitting data to the information terminal, based on the communication state recognized d by the communication recognizing unit; and
a first transmitting unit that transmits the measurement result stored in the first storing unit to the information terminal at the transmission timing determined by the determining unit, and
the information terminal includes:
a first receiving unit that receives the measurement result from the measuring apparatus; and
a first analyzing unit that analyzes the measurement result received by the first receiving unit.

2. The system according to claim 1, wherein
the measuring apparatus further includes:
a user state recognizing unit that recognizes a user state indicating a state of a living body of the user, based on the biological information measured by the sensor, and wherein
the first storing unit stores the user state recognized by the user state recognizing unit as the measurement result.

3. The system according to claim 1, wherein
the measuring apparatus further includes:
a user state recognizing unit that recognizes a user state indicating a state of a living body of the user, based on the biological information measured by the sensor, and wherein
the first storing unit stores the user state recognized by the user state recognizing unit together with the measurement result,
the first transmitting unit transmits the measurement result and the user state to the information terminal at the transmission timing determined by the determining unit, and
the first analyzing unit analyzes the measurement result and the user state.

4. The system according to claim 3, wherein
the first analyzing unit included in the information terminal performs a process that requires a larger amount of computation on the measurement result than a process performed by the user state recognizing unit.

5. The system according to claim 1, wherein
the measuring apparatus further includes a deleting unit that deletes the measurement result from the first storing unit, when the first transmitting unit has transmitted the measurement result.

6. The system according to claim 1, wherein
the sensor measures a body movement of the user,
the communication state recognizing unit recognizes whether communication is possible, based on the body movement measured by the sensor, and
the determining unit judges that a time at which the user is in a state of being able to communicate is the transmission timing.

7. The system according to claim 1, wherein
the communication state recognizing unit recognizes an elapsed period of time since a predetermined time as a current state, and
the determining unit judges that a time at which the elapsed period of time exceeds a predetermined threshold value is the transmission timing.

8. The system according to claim 1, wherein
the communication state recognizing unit judges whether the measuring apparatus is able to communicate with the information terminal, and
the determining unit judges that a time at which the measuring apparatus is able to communicate with the information terminal is the transmission timing.

9. The system according to claim 1, wherein
the measuring apparatus further includes:
a user state recognizing unit that recognizes a user state of the user, based on the biological information measured by the sensor, wherein
the first storing unit stores the user state recognized by the user state recognizing unit together with the measurement result; the measuring apparatus further includes:
a second storing unit that stores a transmission condition that is a predetermined condition and is related to the measurement result;
a judging unit that judges whether the measurement result satisfies the transmission condition stored in the second storing unit; and
a first controlling unit that determines whether at least one of the user state and the measurement result are to be transmitted, based on a judgment result obtained by the judging unit, and wherein
the first transmitting unit transmits the at least one of the user state and the measurement result determined to be transmitted by the first controlling unit, to the information terminal.

10. The system according to claim 9, wherein
the information terminal further includes:
a third storing unit that stores the transmission condition; and
a second transmitting unit that transmits the transmission condition stored in the third storing unit to the measuring apparatus,
the measuring apparatus further includes:
a second receiving unit that receives the transmission condition from the information terminal, and wherein
the second storing unit included in the measuring apparatus stores the transmission condition received by the second receiving unit.

11. The system according to claim 1, further comprising:
a management server that is connected to the information terminal via the Internet and manages the measurement result, wherein
the information terminal further includes a transmitting unit that transmits an analysis result obtained by the first analyzing unit to the management server, and
the management server further includes:
a third receiving unit that receives the analysis result from the information terminal; and
a fourth storing unit that stores the analysis result received by the third receiving unit.

12. The system according to claim 1, wherein
the information terminal further includes:
a call communication unit; and
a fourth transmitting unit that transmits a call receiving notification indicating that the call has received to the measuring apparatus, when the call communication unit has received a call, and wherein
the determining unit included in the measuring apparatus judges that a time when the call receiving notification has been received is not the transmission timing.

13. The system according to claim 1, wherein
the information terminal further includes:
a call communication unit; and
a second controlling unit that disconnects the communication between the information terminal and the measuring apparatus when the call communication unit has received a call, and that re-connects the information terminal to the measuring apparatus when call communication of the call has been finished.

14. The system according to claim 1, wherein
the first analyzing unit included in the information terminal analyzes an asleep/awake state of the user, and
the information terminal further includes:
a call communication unit; and
a third controlling unit that controls a call receiving operation of the call communication unit, based on an analysis result obtained by the first analyzing unit.

15. The system according to claim 14, wherein
the third controlling unit sets the call receiving operation so that a vibrator is used, when the first analyzing unit has judged that the user is in the asleep state.

16. The system according to claim 14, wherein
the third controlling unit sets the call receiving operation so that a ring-tone is used, when the first analyzing unit has judged that the user is in the awake state.

17. The system according to claim 14, further comprising:
a fifth storing unit that stores analysis results, call receiving operations, and call communication partners in correspondence with one another, wherein
the third controlling unit sets the call receiving operation to a call receiving operation in correspondence with a call communication partner and the analysis result obtained at a time when a call has been received, based on a call receiving operation table.

18. An apparatus for measuring biological information of a user, the apparatus comprising:
a sensor that measures the biological information;
a storing unit that stores a measurement result obtained by the sensor;
a communication state recognizing unit that recognizes a current communication state;
a determining unit that determines a transmission timing capable of transmitting data to an information terminal, based on the communication state recognized by the communication state recognizing unit; and
a transmitting unit that transmits the measurement result stored in the storing unit to the information terminal at the transmission timing determined by the determining unit.

19. A measuring method in a biological information measuring system including a measuring apparatus that measures biological information of a user and an information terminal that manages the biological information measured by the measuring apparatus, the measuring method comprising:
measuring the biological information by using a sensor included in the measuring apparatus;
recognizing a current communication state by using the measuring apparatus;
determining a transmission timing capable of transmitting data to the information terminal by using the measuring apparatus, based on the communication state;
transmitting a measurement result stored in a storing unit that stores the measurement result obtained by the sensor by using the measuring apparatus, to the information terminal at the transmission timing;
receiving the measurement result from the measuring apparatus by using the information terminal; and
analyzing the measurement result by using the information terminal.

20. A computer program product having a computer readable medium including programmed instructions for measuring biological information of a user, wherein the instructions, when executed by a computer, cause the computer to perform:
recognizing a current communication state;
determining a transmission timing capable of transmitting data to an information terminal, based on the communication state; and
transmitting a measurement result stored in a storing unit that stores the measurement result obtained by a sensor to the information terminal at the transmission timing.
